(19) United States
(12) Patent Application Publication
(10) Pub. No.: US 2026/0294832 A1
(43) Pub. Date: Oct. 1, 2026

(54) POLYMER NANOPARTICLE COMPOSITIONS FOR NON-VIRAL GENE DELIVERY

(71) Applicant: BATTELLE MEMORIAL INSTITUTE, Columbus, OH (US)

(72) Inventors: Andrea D. McCue, Columbus, OH (US); Kenneth R. Sims, Jr., Columbus, OH (US); Danielle J. Huk, Columbus, OH (US); Jacqueline Grible, Columbus, OH (US); Ashlee J. Colbert, Columbus, OH (US); Emma K. Schmitz, Columbus, OH (US); Anthony D. Duong, Columbus, OH (US)

(21) Appl. No.: 19/630,355

(22) Filed: Mar. 26, 2026

Related U.S. Application Data
(60) Provisional application No. 63/779,211, filed on Mar. 27, 2025.

(51) Int. Cl. A61K 9/51; A61K 31/7105; C08F 293/00; C12N 15/88

(52) U.S. Cl. CPC A61K 9/5138; A61K 31/7105; C08F 293/00; C12N 15/88

(57) ABSTRACT

The disclosure relates to block copolymer nanoparticles for therapeutic delivery of nucleic acids, and methods therefor. More particularly, the invention relates to polymer nanoparticles, such as reversible addition-fragmentation chain transfer (RAFT) polymer compositions, for delivering RNAs.

### RELATED APPLICATIONS

This application claims the benefit of U.S. Provisional Application 63/779,211, filed Mar. 27, 2025, the entire disclosure of which is incorporated herein by reference.

### BACKGROUND

Genetic medicines (including gene therapy, gene silencing, splicing regulators, and nuclease-based gene editors) are poised to produce revolutionary treatments, including vaccines, infectious disease treatments, antimicrobial treatments, antiviral treatments, and most notably, genetic disease treatments. However, the in vivo delivery of these genetic medicine payloads to the specific tissues and cells that need to be treated, while avoiding tissues and cells that can reduce the efficacy or safety of the genetic medicine, poses a significant challenge. Additional challenges include the ability to deliver large genetic payloads or multiple payloads. Adeno-associated viruses (AAVs) are the most widely used tool for genetic medicine delivery, but AAVs are not able to deliver genetic payloads or multiple payloads (such as the clustered regularly interspaced short palindromic repeats (CRISPR)/Cas9 system), and they sometimes trigger unwanted immune responses, including the generation of anti-AAV antibodies, a cell mediated response. Some of the immune responses caused by AAV in patients are potentially fatal immune responses.

Therapeutics based on the CRISPR/Cas9 system have an exceptional potential to treat a number of genetic diseases due to the capability of this system for precise and programmable gene editing. Gene editing and repair using the CRISPR/Cas9 system has two main mechanisms, including non-homologous end joining (NHEJ) which repairs the site of cut by inducing random indel mutation, and homology-directed repair (HDR), which repairs the cut site based on a pre-existing template. Because a pre-designed template can be used for HDR-directed repair, therapies based on this mechanism can be tailored to cure a large number of different genetic diseases. However, the main challenge is that HDR repair requires the delivery of CRISPR/Cas9, small guide RNA (sgRNA) and a donor DNA strand at the same time to a particular location. This requirement becomes particularly limiting for in vivo applications because ensuring co-delivery of multiple large molecules to the same targeted location is currently not feasible. For example, the Cas9 enzyme sequence and guide RNA complex is too large to fit into AAVs.

Thus, there is a need for effective non-viral delivery systems, including gene delivery systems. The current state-of-the-art non-viral gene delivery systems, such as liposomes, have many drawbacks such as poor biocompatibility and the inability to easily engineer or functionalize them. Additional concerns are that such non-viral gene delivery systems are easily degraded by various enzymes as they pass through intracellular or intercellular compartments, and these systems have not been able to package multiple large payloads.

### SUMMARY

In certain aspects, the disclosure is directed to block copolymers (e.g., diblock copolymers). In certain aspects of the present disclosure, a block copolymer comprises
a first block comprising a first homopolymer of monomer units of formula I:

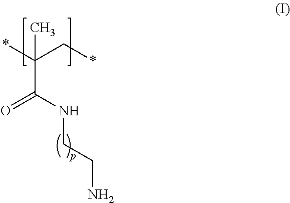

or a salt thereof, wherein:
   p is 0 or an integer selected from 1-3,
   wherein the * represent the connecting points to the rest of the block copolymer; and
a second block comprising a second homopolymer of monomer units of formula II:

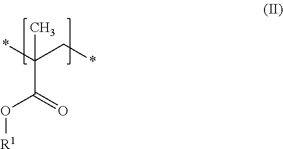

or a salt thereof, wherein:
   R¹ is alkyl; and
wherein each * represents a connecting point to the rest of the block copolymer.

In certain aspects, a block copolymer comprises a first block and a second block, wherein the first block comprises a first homopolymer of poly(2-aminoethyl methacrylamide) p(AEMA) and the second block comprises a second homopolymer of poly (butylmethacrylate) p(BMA) or a homopolymer of poly(methylmethacrylate) p(MMA).

In certain aspects, the copolymer has (i) an overall molecular weight (M_{w}) of about 25 kDa to about 70 kDa, (ii) a theoretical corona to core ratio (CCR) of at least about 5, or (iii) both (i) and (ii).

In certain aspects, the copolymer has (i) an overall molecular weight (Mₙ) of about 15 kDa to about 50 kDa, (ii) a theoretical corona to core ratio (CCR) of at least about 5, or (iii) both (i) and (ii).

In certain aspects, the disclosure relates to a polymer nanoparticle comprising a block copolymer according to the present disclosure.

In certain aspects, the disclosure relates to a composition comprising:
a polymer nanoparticle according to the present disclosure, and
a nucleic acid complexed to the polymer nanoparticle.

In certain aspects, the disclosure relates to a method of treating a disease comprising administering a therapeutically effective amount of a composition according to the present disclosure to a patient in need thereof.

In certain aspects, the disclosure relates to a method of transfecting a cell comprising contacting the cell with an effective amount of a composition according to the present disclosure.

Additional embodiments, features, and advantages of the disclosure will be apparent from the following detailed description and through practice of the disclosure.

### BRIEF DESCRIPTION OF THE DRAWINGS

FIGS. 1A and 1B show Dynamic Light Scattering (DLS) results showing size (FIG. 1A) and polydispersity index values (FIG. 1B) for PNPs 1-3 at multiple concentrations in 1×PBS.

FIGS. 1C and 1D show Dynamic Light Scattering (DLS) results showing size (FIG. 1C) and polydispersity index values (FIG. 1D) for PNPs 4-6 at multiple mass loading ratios of circRNA encoding a fluorescent reporter in 5% glucose solution.

FIG. 2 shows Zeta potential results for PNPs 1-6. ns=not statistically significant and **** p≤0.0001 as determined via One-Way ANOVA with Dunnett's multiple comparisons test.

FIG. 3A shows a heat map of in vitro transfection efficiency (% TE) results for PNPs 1-3 loaded with eGFP circRNA and used to treat HEK293T cells.

FIG. 3B shows a heat map of in vitro transfection efficiency (% TE) results for PNPs 4-6 loaded with eGFP circRNA and used to treat HEK293T cells.

FIGS. 4A and 4B show IVIS images of ICV injection results for PNP1 (FIG. 4A), and PNP2 and PNP3 (FIG. 4B) loaded with mCherry circRNA into wild-type mice at 3 days post-injection. Excised brains of mice labelled “PNP2” and “PNP3” (FIG. 4B, bottom image).

FIG. 5 shows an IVIS image of ICV injection results for PNP2 loaded with Cre mRNA into Ai9 mice at 6 days post-injection.

FIG. 6 shows Ribogreen results for PNPs loaded with eGFP pDNA, eGFP mRNA, and eGFP circRNA loaded at 5 different PNP:cargo weight ratios (30:1, 20:1, 10:1, 6:1, and 4:1).

### DETAILED DESCRIPTION

This disclosure describes compositions of cationic polymers (e.g., diblock copolymers) which self-assemble in aqueous conditions to form polymer nanoparticles (PNPs). In illustrative embodiments, the PNPs efficiently form complexes with nucleic acids. The complexes can then be used to deliver the nucleic acids into cells (e.g., neuronal cells, such as Schwann cells) in vitro and in the brain cortex region in vivo.

Certain embodiments of this disclosure relate to the use of polymer nanoparticle compositions (e.g., a polymer nanoparticle derived from a controlled living/radical polymerization process, such as RAFT copolymers) as a platform with a high degree of tunability in structure and function, opportunities to protect payloads from adverse reactions or degradation by the immune system, and passive cell targeting via surface charge, or particle size. These delivery systems also lend themselves to computer-aided design, and they have suitable pathways to robust, commercial scale manufacturing processes with higher yields and fewer purification steps than viral delivery composition manufacturing processes.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein, the singular forms “a,” “an,” and “the” include plural referents unless the context clearly dictates otherwise. It is further noted that the clauses may be drafted to exclude any optional element. As such, this statement is intended to serve as antecedent basis for use of such exclusive terminology as “solely,” “only” and the like in connection with the recitation of clause elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” may be an approximation of ±10%, ±5%, or ±1%. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2014 (ACD/Labs) or ChemBioDraw Ultra 13.0 (Perkin Elmer).

As used herein and in connection with chemical structures depicting the various embodiments described herein, “*”, “**”, and “ [symbol] ”, each represent a point of covalent attachment of the chemical group or chemical structure in which the identifier is shown to an adjacent chemical group or chemical structure. For example, in a hypothetical chemical structure A-B, where A and B are joined by a covalent bond, in some embodiments, the portion of A-B defined by the group or chemical structure A can be represented by

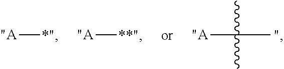

where each of “-*”, “-**”, and

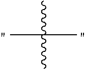

represents a bond to A and the point of covalent bond attachment to B. Alternatively, in some embodiments, the portion of A-B defined by the group or chemical structure B can be represented by

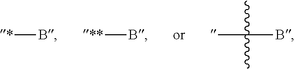

where each of “-*”, “-**”, and

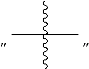

represents a bond to B and the point of covalent bond attachment to A.

As used herein, “molecular weight” refers to weight-average molecular weight (M_{w}) or number-average molecular weight (Mₙ), as indicated herein. Methods of determining molecular weight (M_{w} and Mₙ) of polymers are known in the art. For example, the weight average molecular weight (M_{w}) can be determined by a conventional polystyrene standard curve gel permeation chromatography method (hereinafter referred to as GPC) using multiple narrow distribution polystyrene standard samples relevant to the present invention and preferably between 2 kDa to 1,000 kDa. The number-average molecular weight (Mₙ) can be determined by multiplying the degree of polymerization value by the average molecular weight of the repeating unit (i.e., the molecular weight of the monomer(s) and any end-group (cap) present).

As used herein, “degree of polymerization (DP)” refers to the number of monomer units in a polymer chain, such as in a block of a block copolymer. Methods of determining degree of polymerization (DP) are known in the art. For example, the DP of a copolymer can be calculated by dividing the total number-average molecular weight (Mₙ) by the average molecular weight of the repeat units, or by using NMR integration to determine the ratio of monomer units to end-group (cap) signals.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit disease may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a compound or a composition which reduces the frequency of, or delays the onset of, symptoms of a medical condition in a subject relative to a subject which does not receive the compound or composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance (e.g., a compound, a composition, or an agent) to a subject can be carried out using one of a variety of methods known to those skilled in the art. For example, a substance can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A substance (e.g., a compound, a composition, or agent) can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance (e.g., a compound, a composition, or an agent) to a subject will also depend, for example, on the age and/or the physical condition of the subject and the chemical and biological properties of the compound or agent (e.g., solubility, digestibility, bioavailability, stability and toxicity). In some embodiments, a substance (e.g., a compound, a composition, or an agent) is administered orally, e.g., to a subject by ingestion. In some embodiments, the orally administered substance is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

### Definitions

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer. The term “alkylene” refers to a straight- or branched-chain divalent hydrocarbon group. In some embodiments, it can be advantageous to limit the number of atoms in an “alkyl” or “alkylene” to a specific range of atoms, such as C₁-C₂₀ alkyl or C₁-C₂₀ alkylene, C₁-C₁₂ alkyl or C₁-C₁₂ alkylene, or C₁-C₆ alkyl or C₁-C₆ alkylene. Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. Examples of alkylene groups include methylene (—CH₂—), ethylene ((—CH₂—)₂), n-propylene ((—CH₂—)₃), iso-propylene ((—C(H)(CH₃)CH₂—)), n-butylene ((—CH₂—)₄), and the like. It will be appreciated that an alkyl or alkylene group can be unsubstituted or substituted as described herein. An alkyl or alkylene group can be substituted with any of the substituents in the various embodiments described herein, including one or more of such substituents. Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “alkenyl” refers to a straight- or branched-chain mono-valent hydrocarbon group having one or more double bonds. In some embodiments, it can be advantageous to limit the number of atoms in an “alkenyl” to a specific range of atoms, such as C₂-C₂₀ alkenyl, C₂-C₁₂ alkenyl, or C₂-C₆ alkenyl. Examples of alkenyl groups include ethenyl (or vinyl), allyl, and but-3-en-1-yl. Included within this term are cis and trans isomers and mixtures thereof. It will be appreciated that an alkenyl can be unsubstituted or substituted as described herein. An alkenyl group can be substituted with any of the substituents in the various embodiments described herein, including one or more of such substituents.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀ alkyl indicates a hydrogen where the group is in a terminal position, a bond if internal. A C₁₋₆ alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

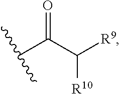

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

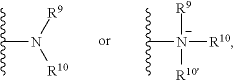

wherein R⁹, R¹⁰, and R¹⁰, each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “carboxy”, as used herein, refers to a group represented by the formula —CO₂H.

The term “ester”, as used herein, refers to a group —C(O)OR⁸ wherein R⁸ represents a hydrocarbyl group.

The term “ketone”, as used herein, refers to a group —C(O)R⁷ wherein R⁷ represents a hydrocarbyl group (e.g., alkyl, aryl, heteroaryl).

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

The term “substituted” refers to moieties having substituents replacing a hydrogen on one or more atoms (e.g., carbon atoms) of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

It is understood that substituents and substitution patterns on the compounds of the present disclosure can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

The nomenclature “(ATOM)ᵢ-(ATOM)ⱼ” with j>i, when applied herein to a class of substituents, is meant to refer to embodiments of this disclosure for which each and every one of the number of atom members, from i to j including i and j, is independently realized. By way of example, the term C₁-C₃ refers independently to embodiments that have one carbon member (C₁), embodiments that have two carbon members (C₂), and embodiments that have three carbon members (C₃). The term “modulate” as used herein includes the inhibition or suppression of a function or activity (such as cell proliferation) as well as the enhancement of a function or activity.

“Acceptable salt” or “salt” is used herein to refer to an acid addition salt or a basic addition salt which is suitable for or compatible with the compounds or a desired treatment.

The disclosure also includes acceptable salts (e.g., pharmaceutically acceptable salts) of the block copolymers described herein, preferably of those described above and of the specific compounds exemplified herein, and pharmaceutical compositions comprising such salts, and methods of using such salts.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

It will be understood that the chemical entities described herein, can exist as a salt of a free acid or base of a compound represented herein and an inorganic or organic counter ion. Illustratively, the salt can be formed during the manufacture of the compound described herein (e.g., a salt or a pharmaceutically acceptable salt) or a compound described herein can be substituted to provide a salt for further manufacture, formulation, or administration reasons. As illustrated herein, certain compounds include a “W^{⊖},” wherein “W^{⊖}” is an inorganic counter ion (e.g., an inorganic anion) or an organic counter ion (e.g., an organic anion). In certain embodiments, W^{⊖} is an anion that is complexed with a cation of a compound of the disclosure to form a pharmaceutically acceptable salt.

The term “acceptable acid addition salt” as used herein means any non-toxic organic or inorganic salt of any base compounds of the present disclosure. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of the present disclosure are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of the present disclosure for laboratory use, or for subsequent conversion to a pharmaceutically acceptable acid addition salt.

The term “acceptable basic addition salt” as used herein means any non-toxic organic or inorganic base addition salt of any acid compounds represented by compounds or polymers of the present disclosure or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The term “inorganic counter ion” represents an inorganic ion that accompanies an ionic species in order to maintain electric neutrality. An inorganic counter ion may represent an anion or cation. An inorganic counterion may accompany a free acid or base of a compound represented herein. An inorganic ion may form by a reaction of an inorganic base or inorganic acid and a compound described herein that possesses a sufficiently acidic group, a sufficiently basic group, both types of functional groups, or more than one of each type.

The term “organic counter ion” represents an organic counter ion that accompanies an ionic species in order to maintain electric neutrality. The organic ion may represent an anion or cation. An organic counter ion may accompany a free acid or base of a compound represented herein. An organic ion may form by a reaction of an organic base or organic acid and a compound described herein that possesses a sufficiently acidic group, a sufficiently basic group, both types of functional groups, or more than one of each type.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a block copolymer that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

As used herein and in connection with chemical structures depicting the various embodiments described herein, a polymer may be arranged in any linear or branched configuration. For example, a polymer comprising one or more monomer units may be arranged as a homopolymer, a block copolymer, a statistical copolymer, a random copolymer, or an alternate copolymer of different monomers linked together in an alternating fashion. In one embodiment, the polymers described herein may include repeating blocks, such as in a block copolymer, of units selected from formula I and formula II.

The term “block copolymer” is known in the art, and a representative definition is that a block copolymer is a polymer comprising molecules in which there is a linear arrangement of blocks, for example a block A linearly connected to a block B, where each of the blocks (e.g., block A and block B) comprises units derived from a characteristic species or combination of species of monomer such that at least one difference exists between the species of monomer(s) of the different blocks (e.g., the monomer species or combination of species of block A is different than the monomer species or combination of species of block B. Illustratively, a block polymer may be represented by a formula including a “-b-” identifier between each block. For example, a block polymer comprising a first block of a AEMA homopolymer (p(AEMA)) and a second block of a BMA homopolymer (p(BMA)) may be represented by the formula: p(AEMA)-b-p(BMA).

The term “statistical copolymer” is known in the art, and a representative definition is that a statistical copolymer can be a copolymer composed of monomers that form a sequence based on a statistical rule (e.g., Markovian statistics).

The term “random copolymer” is known in the art, and a representative definition is that a random copolymer describes a copolymer where the probability of finding a given type monomer residue at a particular point in the chain is equal to the mole fraction of that monomer residue in the chain and is independent of the neighboring units in the chain.

The term “alternate copolymer” is known in the art, and a representative definition is that an alternate copolymer describes a copolymer of monomers sequentially linked in a uniform pattern.

“Nucleotide” as used herein is a molecule that contains a base moiety, a sugar moiety, and a phosphate moiety. Nucleotides can be linked together through their phosphate moieties and sugar moieties creating an internucleoside linkage. The term “oligonucleotide” is sometimes used to refer to a molecule that contains two or more nucleotides linked together. The base moiety of a nucleotide can be adenine-9-yl (A), cytosine-1-yl (C), guanine-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide that contains some type of modification to the base, sugar, and/or phosphate moieties. Modifications to nucleotides are well known in the art and would include, for example, 5-methylcytosine (5-me-C), 5 hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but are linked together through a moiety other than a phosphate moiety. Nucleotide substitutes are able to conform to a double helix type structure when interacting with the appropriate target nucleic acid.

The term “polynucleotide,” as used herein, means a molecule including one or more nucleic acid subunits, or nucleotides, and can be used interchangeably with “nucleic acid” or “oligonucleotide”. A polynucleotide can include one or more nucleotides selected from adenosine (A), cytosine (C), guanine (G), thymine (T) and uracil (U), or variants thereof. A nucleotide can include a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. Ribonucleotides are nucleotides in which the sugar is ribose. Polyribonucleotides or ribonucleic acids, or RNA, can refer to macromolecules that include multiple ribonucleotides that are polymerized via phosphodiester bonds. Deoxyribonucleotides are nucleotides in which the sugar is deoxyribose. As used herein, a polyribonucleotide sequence that recites thymine (T) is understood to represent uracil (U).

“Polydeoxyribonucleotides,” “deoxyribonucleic acids,” and “DNA” mean macromolecules that include multiple deoxyribonucleotides that are polymerized via phosphodiester bonds. “Polyribonucleotides,” “ribonucleic acids,” and “RNA” mean macromolecules that include multiple ribonucleotides that are polymerized via phosphodiester bonds. A nucleotide can be a nucleoside monophosphate or a nucleoside polyphosphate. A nucleotide means a deoxyribonucleoside polyphosphate, such as, e.g., a deoxyribonucleoside triphosphate (dNTP), which can be selected from deoxyadenosine triphosphate (dATP), deoxycytidine triphosphate (dCTP), deoxyguanosine triphosphate (dGTP), deoxyuridine triphosphate (dUTP) and deoxythymidine triphosphate (dTTP) dNTPs, and may include detectable tags, such as protein tags, luminescent tags or markers (e.g., fluorophores). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof).

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, mRNA, circRNA, cDNA, gDNA, ssDNA, dsDNA or any combination thereof.

The term “vector” or “construct” designates a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “operably linked to” refers to the functional relationship of a nucleic acid with another nucleic acid sequence. Promoters, enhancers, transcriptional and translational stop sites, and other signal sequences are examples of nucleic acid sequences that can operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA. DNA and RNA can be synthesized naturally (e.g. by DNA replication or transcription of DNA or RNA, respectively). DNA and RNA can also be chemically synthesized. RNA can be post-transcriptionally modified. The terms “target mRNA” and “target transcript,” “target sequence” are synonymous as used herein.

As used herein, “target sequence” refers to a contiguous portion of the nucleotide sequence of a nucleic acid molecule formed during the transcription of a gene, including mRNA that is a product of RNA processing of a primary transcription product.

“Targeting” an oligomeric compound to a particular nucleic acid molecule, in the context of this invention, can be a multistep process. The process usually begins with the identification of a target nucleic acid whose function is to be modulated. This target nucleic acid may be, for example, a cellular gene (or mRNA transcribed from the gene) whose expression is associated with a particular disorder or disease state, or a nucleic acid molecule from an infectious agent.

The targeting process usually also includes determination of at least one target region, segment, or site within the target nucleic acid for the antisense interaction to occur such that the desired effect, e.g., modulation of expression, will result. Within the context of the present invention, the term “region” is defined as a portion of the target nucleic acid having at least one identifiable structure, function, or characteristic. Within regions of target nucleic acids are segments. “Segments” are defined as smaller or sub-portions of regions within a target nucleic acid. “Sites,” as used in the present invention, are defined as positions within a target nucleic acid.

The term “RNAi” as used herein refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but are not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein).

The term “RNAi” and “RNA interfering” with respect to an agent of the invention, are used interchangeably herein. RNAi molecules are typically comprised of a sequence of nucleic acids or nucleic acid analogs, specific for a target gene. A nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA).

As used herein, “shRNA” or “small hairpin RNA” (also called stem loop) is a type of RNAi. shRNAs may be composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow. shRNAs functions as RNAi but are further defined in that shRNA species are double stranded hairpin-like structure for increased stability. These shRNAs, as well as other such agents described herein, can be contained in plasmids, retroviruses, and lentiviruses and expressed from, for example, the pol III U6 promoter, or another promoter.

### REPRESENTATIVE EMBODIMENTS

This disclosure describes compositions of cationic polymers (e.g., diblock copolymers) which self-assemble in aqueous conditions to form polymer nanoparticles (PNPs). In illustrative embodiments, the PNPs can complex nucleic acids and deliver the complexed nucleic acids to a desired location. In certain embodiments, the composition delivers RNA to the brain, for example in a mammal such as a mouse, upon local administration.

In certain embodiments, a block copolymer comprises a first block and a second block. The first block may comprise a homopolymer of poly (aminoethyl methacrylamide) p(AEMA). The second block may comprise a homopolymer of poly (butylmethacrylate) p(BMA). The second block may comprise a homopolymer of poly(methylmethacrylate) p(MMA). In certain preferred embodiments, the block copolymer is a diblock copolymer.

In certain embodiments, a block copolymer comprises a first block and a second block that is different from the first block. In certain embodiments, the first block is a homopolymer and the second block is a homopolymer. The first block can be a homopolymer of monomer units, wherein each monomer unit is represented by formula I:

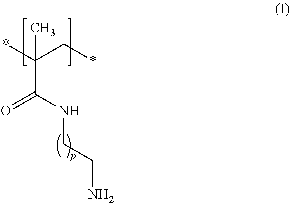

or a salt thereof, wherein:
   p is 0 or an integer selected from 1-3, and
   wherein each * individually represents a point of covalent attachment to the rest of the block copolymer; and
the second block can be a homopolymer of monomer units, wherein each monomer unit is represented by formula II:

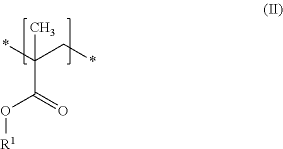

or a salt thereof, wherein:
   R¹ is alkyl, and
   wherein each * individually represents a point of covalent attachment to the rest of the block copolymer.

In one aspect, a block copolymer comprises a first block and a second block that is different from the first block. The first block can be a homopolymer of monomer units, wherein each monomer unit is represented by formula I:

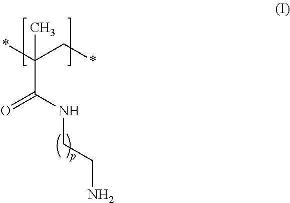

or a salt thereof, wherein p is 0 or an integer selected from 1-3 and each * individually represents a point of covalent attachment to the rest of the block copolymer.

In some embodiments, p is 0. In some embodiments, p is an integer selected from 1-3, and is preferably 1.

In some embodiments, the second block is a homopolymer of monomer units, wherein each monomer unit is represented by formula II:

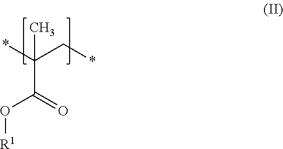

or a salt thereof, wherein:

R¹ is alkyl (e.g., C₁-C₆ alkyl, and wherein each * individually represents a point of covalent attachment to the rest of the block copolymer.

In some embodiments, the monomer unit of the first block is preferably

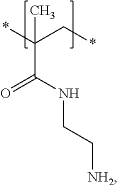

or a salt thereof, wherein each * represents a point of covalent attachment to the rest of the block copolymer. Illustratively, the monomer unit of the first block is derivable from the polymerization of 2-aminoethylmethacrylamide (AEMA) (i.e.,

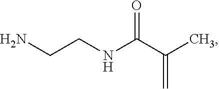

preferably the HCl salt thereof).

In certain preferred embodiments, the second block comprises a homopolymer. For example, in some embodiments, the second block comprises a homopolymer of BMA or a homopolymer of MMA.

In some embodiments, R¹ is C₁-C₆ alkyl. In some embodiments, R¹ is C₃-C₆ alkyl, such as a straight chain C₃-C₆ alkyl, which may be optionally substituted as described herein. In certain preferred embodiments, R¹ is C₃-C₆ alkyl (e.g., C₄ alkyl such as n-butyl) and is unsubstituted.

In some embodiments, the monomer unit of the second block is preferably

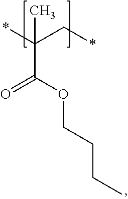

or a salt thereof, wherein each * represents a point of covalent attachment to the rest of the block copolymer. In some embodiments, the monomer unit of the first block is derivable from the polymerization of butylmethacrylate (BMA)

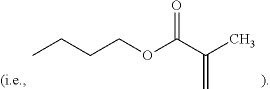

In some embodiments, R¹ is C₁-C₃ alkyl, such as a straight chain C₁-C₃ alkyl, which may be optionally substituted as described herein. In certain preferred embodiments, R¹ is C₁-C₃ alkyl (e.g., C₁ alkyl such as methyl) and is unsubstituted. In some embodiments, the monomer unit of the first block is derivable from the polymerization of methylmethacrylate (MMA)

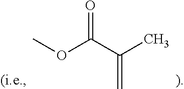

In another aspect, a block copolymer comprises a first block and a second block, wherein:
the first block comprises a first homopolymer of poly(aminoethyl methacrylamide) p(AEMA), and
the second block comprises a second homopolymer of poly(butylmethacrylate) p(BMA).

In another aspect, a block copolymer comprises a first block and a second block, wherein:
the first block comprises a first homopolymer of poly(aminoethyl methacrylamide) p(AEMA), and
the second block comprises a second homopolymer of poly(methylmethacrylate) p(MMA).

In some embodiments, a block copolymer is selected from p(AEMA)-b-p(BMA), and p(AEMA)-b-p(MMA), preferably p(AEMA)-b-p(BMA).

In some embodiments, the block copolymer comprises at least about 70% of the first block by weight. For example, the block copolymer may comprise at least about 80% of the first block by weight. In some embodiments, the block copolymer comprises about 70% to about 99% of the first block by weight. For example, the block copolymer may comprise about 80% to about 99% of the first block by weight. In some embodiments, the block copolymer comprises about 85% to about 99% of the first block by weight.

The block copolymer described herein comprises a ratio of monomer units of formula I (e.g., monomer units of AEMA) to monomer units of formula II (e.g., monomer units of BMA). In some embodiments, the ratio of monomer units of formula I to monomer units of formula II is about 80:20 to about 99:1. In some embodiments, the ratio of monomer units of formula I to monomer units of formula II is about 85:15 to about 99:1. In some embodiments, the ratio of monomer units of formula I to monomer units of formula II is about 90:10 to about 99:1. In some embodiments, the ratio of monomer units of formula I to monomer units of formula II is about 95:5 to about 99:1.

In some embodiments, the first block has a degree of polymerization (DP), for example a theoretical DP, of at least about 125. In some embodiments, the DP of the first block is about 125 to about 230, about 140 to about 230, or about 150 to about 230. In some embodiments, the DP of the first block is about 125, about 130, about 135, about 140, about 145, about 150, about 155, about 160, about 165, about 170, about 175, about 180, about 185, about 190, about 195, about 200, about 205, about 210, about 220, or about 230. In some preferred embodiments, the DP of the first block is about 160 to about 180.

In some embodiments, the second block has a degree of polymerization (DP), for example a theoretical DP, less than the first block. For example, in some embodiments, the DP of the second block is about 10 to about 130, about 10 to about 120, about 10 to about 100, about 10 to about 90, about 10 to about 80, about 10 to about 70, about 10 to about 60, about 10 to about 50, about 10 to about 40, or about 15 to about 40. In some embodiments, the DP of the second block is about 10, about 15, about 20, about 25, about 30, about 35, about 40, about 45, about 50, about 55, about 60, about 65, about 70, about 75, about 80, about 85, about 90, about 95, about 100, about 110, about 120, or about 130. In some preferred embodiments, the DP of the second block is about 20.

In some embodiments, the block copolymer has an overall degree of polymerization (DP), which is defined as the calculated from the sum of the degrees of polymerization of the individual blocks. In some embodiments, the block copolymer has an overall degree of polymerization of about 100 to about 300. For example, the block copolymer may have an overall degree of polymerization of about 150 to about 250.

A block copolymer may have a theoretical corona-to-core molecular weight ratio (CCR), which is defined as the calculated ratio of the first block theoretical molecular weight to the second block theoretical molecular weight. The theoretical CCR may be based on weight-average molecular weight (M_{w}) or number-average molecular weight (Mₙ). In some embodiments, the block copolymer has a theoretical CCR of at least about 5 or at least about 7. In some embodiments, the theoretical CCR is about 5 to about 40, about 7 to about 40, about 5 to about 25, about 7 to about 25, about 5 to about 10, or about 7 to about 10. In some embodiments, the block copolymer has a theoretical CCR of about 0.1 to about 40. In some embodiments, the block copolymer has a theoretical CCR of about 1 to about 40.

In some embodiments, the block copolymer has a theoretical CCR (based on Mₙ) of at least about 5 or at least about 7. In some embodiments, the block copolymer has a theoretical CCR (based on Mₙ) of at least about 7. In some embodiments, the block copolymer has a theoretical CCR (based on Mₙ) of at least about 7.5. In some embodiments, the block copolymer has a theoretical CCR (based on Mₙ) of at least about 8. In some embodiments, the block copolymer has a theoretical CCR (based on Mₙ) of about 5 to about 40. For example, the theoretical CCR (based on Mₙ) may be about 5 to about 40, about 7 to about 40, about 5 to about 25, about 7 to about 25, about 5 to about 10, or about 7 to about 10. In some embodiments, the block copolymer has a theoretical CCR (based on Mₙ) of about 5, about 5.5, about 6, about 6.5, about 7, about 7.5, about 8, about 8.5, about 9, about 9.5, or about 10. In some embodiments, the block copolymer has a theoretical CCR (based on Mₙ) of about 5 to about 10. In some embodiments, the block copolymer has a theoretical CCR (based on Mₙ) of about 7 to about 10. In some embodiments, the block copolymer has a theoretical CCR (based on Mₙ) of about 7.5 to about 10. In some embodiments, the block copolymer has a theoretical CCR (based on Mₙ) of about 8 to about 10 (e.g., about 8.5 to about 9.5).

In some embodiments, the block copolymer has a theoretical CCR (based on M_{w}) of at least about 0.1 or at least about 1. In some embodiments, the block copolymer has a theoretical CCR (based on M_{w}) of about 0.1 to about 40, such as about 1 to about 40. For example, the theoretical CCR (based on M_{w}) may be about 0.1 to about 40, about 0.5 to about 40, about 1 to about 40, about 0.1 to about 25, about 0.5 to about 25, about 1 to about 25, about 0.1 to about 10, about 0.5 to about 10, or about 1 to about 10. In some embodiments, the block copolymer has a theoretical CCR (based on M_{w}) of about 0.5, about 1, about 1.5, about 2, about 2.5, about 3, about 3.5, about 4, about 4.5, or about 5. In some embodiments, the block copolymer has a theoretical CCR (based on M_{w}) of about 0.1 to about 5, such as about 0.5 to about 5 (e.g., about 0.6 to about 3.4).

In some embodiments, the first block has a molecular weight (Mₙ) of at least about 15,000 Da. In some embodiments, the first block has a molecular weight (Mₙ) of about 15,000 Da to about 50,000 Da. For example, the first block may be about 15,000 Da to about 40,000 Da, about 15,000 Da to about 30,000 Da, about 15,000 Da to about 25,000 Da, or about 15,000 Da to about 20,000 Da. In some embodiments, the first block is about 15,000 Da to about 40,000 Da. In some embodiments, the first block is about 15,000 Da to about 25,000 Da.

In some embodiments, the first block has a molecular weight (M_{w}) of at least about 10,000 Da, at least about 15,000 Da, at least about 20,000 Da, or at least about 25,000 Da. In some embodiments, the first block has a molecular weight (M_{w}) of about 10,000 Da to about 70,000 Da. For example, the first block may be about 15,000 Da to about 70,000 Da, about 20,000 Da to about 70,000 Da, about 25,000 Da to about 70,000 Da, about 30,000 Da to about 70,000 Da, about 10,000 Da to about 60,000 Da, about 15,000 Da to about 60,000 Da, about 20,000 Da to about 60,000 Da, about 25,000 Da to about 60,000 Da, or about 30,000 Da to about 60,000 Da. In some embodiments, the first block is about 10,000 Da to about 50,000 Da, about 15,000 Da to about 50,000 Da, about 20,000 Da to about 50,000 Da, about 25,000 Da to about 50,000 Da, about 10,000 Da to about 40,000 Da, about 15,000 Da to about 40,000 Da, about 20,000 Da to about 40,000 Da, or about 25,000 Da to about 40,000 Da. In some embodiments, the first block is about 20,000 Da to about 40,000 Da.

In some embodiments, the first block has a molecular weight (Mₙ) of at least about 15 kDa, and the block copolymer comprises a ratio of monomer units of formula I to monomer units of formula II is about 80:20 to about 99:1. In some embodiments, the first block has a molecular weight (Mₙ) of at least about 15 kDa, and the block copolymer comprises a ratio of monomer units of formula I to monomer units of formula II is about 85:15 to about 99:1 In some embodiments, the first block has a molecular weight (Mₙ) of at least about 15 kDa, and the block copolymer comprises a ratio of monomer units of formula I to monomer units of formula II is about 90:10 to about 99:1. In some embodiments, the first block has a molecular weight (Mₙ) of at least about 15 kDa, and the block copolymer comprises a ratio of monomer units of formula I to monomer units of formula II is about 95:5 to about 99:1.

In some embodiments, the first block has a molecular weight (M_{w}) of at least about 20 kDa, and the block copolymer comprises a ratio of monomer units of formula I to monomer units of formula II is about 80:20 to about 99:1. In some embodiments, the first block has a molecular weight (M_{w}) of at least about 20 kDa, and the block copolymer comprises a ratio of monomer units of formula I to monomer units of formula II is about 85:15 to about 99:1. In some embodiments, the first block has a molecular weight (M_{w}) of at least about 20 kDa, and the block copolymer comprises a ratio of monomer units of formula I to monomer units of formula II is about 90:10 to about 99:1. In some embodiments, the first block has a molecular weight (M_{w}) of at least about 20 kDa, and the block copolymer comprises a ratio of monomer units of formula I to monomer units of formula II is about 95:5 to about 99:1.

In some embodiments, the first block has a molecular weight (Mₙ) of at least about 15 kDa, and the block copolymer comprises a theoretical CCR of at least about 5. In some embodiments, the first block has a molecular weight (Mₙ) of at least about 15 kDa, and the block copolymer comprises a theoretical CCR of at least about 7. In some embodiments, the first block has a molecular weight (Mₙ) of at least about 15 kDa, and the block copolymer comprises a theoretical CCR of at least about 7.5. In some embodiments, the first block has a molecular weight (Mₙ) of at least about 15 kDa, and the block copolymer comprises a theoretical CCR of at least about 8.

In some embodiments, the first block has a molecular weight (M_{w}) of at least about 20 kDa, and the block copolymer comprises a theoretical CCR of at least about 5. In some embodiments, the first block has a molecular weight (M_{w}) of at least about 20 kDa, and the block copolymer comprises a theoretical CCR of at least about 7. In some embodiments, the first block has a molecular weight (M_{w}) of at least about 20 kDa, and the block copolymer comprises a theoretical CCR of at least about 7.5. In some embodiments, the first block has a molecular weight (M_{w}) of at least about 20 kDa, and the block copolymer comprises a theoretical CCR of at least about 8.

In some embodiments, the second block has a molecular weight (Mₙ) of about 1,000 Da to about 5,000 Da, such as about 1,000 Da to about 4,000 Da.

In some embodiments, the second block has a molecular weight (M_{w}) of about 1,000 Da to about 50,000 Da. For example, the second block may be about 1,000 Da to about 45,000 Da, about 1,000 Da to about 40,000 Da, about 1,000 Da to about 35,000 Da, about 1,000 Da to about 30,000 Da, about 1,000 Da to about 25,000 Da, about 1,000 Da to about 20,000 Da, about 1,000 Da to about 15,000 Da, about 1,000 Da to about 12,000 Da, about 1,000 Da to about 10,000 Da, about 1,000 Da to about 7,000 Da, or about 1,000 Da to about 5,000 Da. In some embodiments, the second block is about 5,000 Da to about 15,000 Da. In some embodiments, the second block is about 35,000 Da to about 45,000 Da.

In some embodiments, a block polymer as described herein has an overall molecular weight (Mₙ) (i.e., the total of all blocks) in the range of about 5 kDa to about 75 kDa, about 5 kDa to about 60 kDa, about 5 kDa to about 50 kDa, about 10 kDa to about 50 kDa, about 15 kDa to about 50 kDa, about 20 kDa to about 50 kDa, about 30 kDa to about 50 kDa, about 5 kDa to about 25 kDa, about 10 kDa to about 25 kDa, about 15 kDa to about 25 kDa, or about 20 kDa to about 25 kDa. In some embodiments, a block polymer as described herein has an overall molecular weight (Mₙ) in the range of about 15 kDa to about 60 kDa, such as about 15 kDa to about 50 kDa. In some embodiments, a block polymer as described herein has an overall molecular weight (Mₙ) in the range of about 15 kDa to about 25 kDa.

In some embodiments, a block polymer as described herein has an overall molecular weight (M_{w}) (i.e., the total of all blocks) in the range of about 5 kDa to about 100 kDa, about 5 kDa to about 75 kDa, about 5 kDa to about 60 kDa, about 10 kDa to about 60 kDa, about 15 kDa to about 60 kDa, about 20 kDa to about 60 kDa, about 30 kDa to about 60 kDa, about 30 kDa to about 55 kDa, or about 35 kDa to about 55 kDa. In some embodiments, a block polymer has an overall molecular weight (M_{w}) in the range of about 25 kDa to about 75 kDa, about 25 kDa to about 70 kDa, about 25 kDa to about 60 kDa, about 30 kDa to about 75 kDa, about 30 kDa to about 70 kDa, or about 30 kDa to about 60 kDa. In some embodiments, a block polymer has an overall molecular weight (M_{w}) in the range of about 25 kDa to about 70 kDa, such as about 25 kDa to about 60 kDa. In some embodiments, a block polymer has an overall molecular weight (M_{w}) in the range of about 30 kDa to about 70 kDa, such as about 30 kDa to about 60 kDa. In certain embodiments, the copolymer has an overall molecular weight (M_{w}) of at least about 25 kDa or at least about 30 kDa. In certain embodiments, the copolymer has an overall molecular weight (M_{w}) of less than about 75 kDa or less than about 70 kDa.

In some embodiments, the block copolymer has a molecular weight (Mₙ) of at least about 15 kDa, and the block copolymer comprises a ratio of monomer units of formula I to monomer units of formula II is about 80:20 to about 99:1. In some embodiments, the block copolymer has a molecular weight (Mₙ) of at least about 15 kDa, and the block copolymer comprises a ratio of monomer units of formula I to monomer units of formula II is about 85:15 to about 99:1. In some embodiments, the block copolymer has a molecular weight (Mₙ) of at least about 15 kDa, and the block copolymer comprises a ratio of monomer units of formula I to monomer units of formula II is about 90:10 to about 99:1. In some embodiments, the block copolymer has a molecular weight (Mₙ) of at least about 15 kDa, and the block copolymer comprises a ratio of monomer units of formula I to monomer units of formula II is about 95:5 to about 99:1.

In some embodiments, the block copolymer has a molecular weight (M_{w}) of at least about 25 kDa, and the block copolymer comprises a ratio of monomer units of formula I to monomer units of formula II is about 80:20 to about 99:1. In some embodiments, the block copolymer has a molecular weight (M_{w}) of at least about 25 kDa, and the block copolymer comprises a ratio of monomer units of formula I to monomer units of formula II is about 85:15 to about 99:1. In some embodiments, the block copolymer has a molecular weight (M_{w}) of at least about 25 kDa, and the block copolymer comprises a ratio of monomer units of formula I to monomer units of formula II is about 90:10 to about 99:1. In some embodiments, the block copolymer has a molecular weight (M_{w}) of at least about 25 kDa, and the block copolymer comprises a ratio of monomer units of formula I to monomer units of formula II is about 95:5 to about 99:1.

In some embodiments, the block copolymer has a molecular weight (Mₙ) of at least about 15 kDa, and the block copolymer comprises a theoretical CCR of at least about 5. In some embodiments, the block copolymer has a molecular weight (Mₙ) of at least about 15 kDa, and the block copolymer comprises a theoretical CCR of at least about 7. In some embodiments, the block copolymer has a molecular weight (Mₙ) of at least about 15 kDa, and the block copolymer comprises a theoretical CCR of at least about 7.5. In some embodiments, the block copolymer has a molecular weight (Mₙ) of at least about 15 kDa, and the block copolymer comprises a theoretical CCR of at least about 8.

In some embodiments, the block copolymer has a molecular weight (M_{w}) of at least about 25 kDa, and the block copolymer comprises a theoretical CCR of at least about 5. In some embodiments, the block copolymer has a molecular weight (M_{w}) of at least about 25 kDa, and the block copolymer comprises a theoretical CCR of at least about 7. In some embodiments, the block copolymer has a molecular weight (M_{w}) of at least about 25 kDa, and the block copolymer comprises a theoretical CCR of at least about 7.5. In some embodiments, the block copolymer has a molecular weight (M_{w}) of at least about 25 kDa, and the block copolymer comprises a theoretical CCR of at least about 8.

In some embodiments, block copolymers as prepared herein can be described by the following structure:

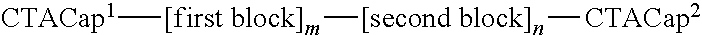

where each CTACap (e.g., CTACap¹ and CTACap²) is a capping unit derived from the chain transfer agent(s) used in the process for preparing the RAFT copolymer. The CTA used for preparing each of the first block and the second block can be the same or different. In some embodiments, the CTA used to prepare each the first block and the second block is the same (e.g., macroCTA). In some embodiments, the CTA used to prepare each of the first block and the second block is different. In some embodiments, the CTA used to prepare one or both of the first block and the second block comprises a functional group for the covalent attachment of a biomolecule, drug, or label to the block copolymer. In some embodiments, the covalent attachment can be via an ester or an amide bond. In some embodiments, the covalent attachment can be via EDC-NHS chemistry.

In some embodiments, the first block comprises a cap of formula:

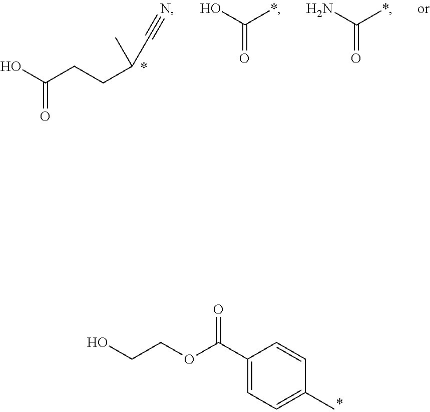

or a salt thereof, wherein * represents a point of covalent attachment to the first block. In certain preferred embodiments, the cap is

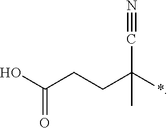

In some embodiments, the second capping unit is of formula

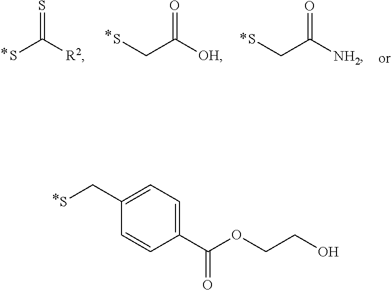

or a salt thereof, wherein * represents a point of covalent attachment to the second block, and R² is —SC₂-C₁₂ alkyl or C₆H₅, and is preferably

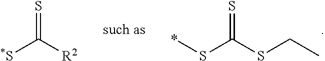

In some embodiments, a polymer nanoparticle comprises a block copolymer according to the present disclosure. In some embodiments, the block copolymer self-assembles into the nanoparticle.

In some embodiments, the nanoparticle has a hydrodynamic diameter (e.g., Z-averaged hydrodynamic diameter) of about 100 nm to about 500 nm, about 150 nm to about 500 nm, about 150 nm to about 400 nm, or about 150 nm to about 350 nm, as measured by DLS.

The hydrodynamic diameter can be measured according to the methods of the present disclosure, and may be, for example determined at values of about 0.5 mg/ml to about 7 mg/ml nanoparticle. In some embodiments, the hydrodynamic diameter is about 150 nm to about 500 nm, when measured at about 0.5 mg/ml to about 7 mg/ml (e.g., at about 0.5 mg/ml, about 2 mg/ml, or about 6 mg/ml) nanoparticle. In certain embodiments, the hydrodynamic diameter is about 200 nm to about 350 nm, when measured at about 0.5 mg/ml to about 7 mg/ml (e.g., at about 0.5 mg/ml, about 2 mg/ml, or about 6 mg/ml) nanoparticle.

The hydrodynamic diameter can be measured according to the methods of the present disclosure, and may be, for example determined at loading ratios of nanoparticle to nucleic acid. In certain embodiments, the hydrodynamic diameter is about 150 nm to about 500 nm, when measured at a loading ratio of about 1:1 to about 30:1 (e.g., about 5:1, about 10:1, or about 20:1). In certain embodiments, the hydrodynamic diameter is about 200 nm to about 350 nm, when measured at a loading ratio of about 1:1 to about 30:1 (e.g., about 5:1, about 10:1, or about 20:1).

In certain embodiments, the nanoparticle is monodispersed, as measured using dynamic light scattering (DLS). In some embodiments, the nanoparticle has a PDI of about 0.05 to about 0.5, as measured by DLS. In certain embodiments, the nanoparticle has a polydispersity index (PDI) of lower than about 0.5 or lower than about 0.3. For example, the nanoparticle can have a PDI of about 0.05 to about 0.5, about 0.05 to about 0.3, or about 0.05 to about 0.2.

The PDI values described herein can be measured according to the methods of the present disclosure, and may be, for example determined at values of about 0.5 mg/ml to about 7 mg/ml nanoparticle. In certain embodiments, the PDI is less than about 0.5 when measured at about 0.5 mg/ml to about 7 mg/ml (e.g., at about 0.5 mg/ml, about 2 mg/ml, or about 6 mg/ml) nanoparticle. In certain embodiments, the PDI is less than about 0.3 when measured at about 0.5 mg/ml to about 7 mg/ml (e.g., at about 0.5 mg/ml, about 2 mg/ml, or about 6 mg/ml) nanoparticle.

The PDI values described herein can be measured according to the methods of the present disclosure, and may be, for example determined at loading ratios of nanoparticle to nucleic acid. In certain embodiments, the PDI is less than about 0.5 when measured at a loading ratio of about 1:1 to about 30:1 (e.g., about 5:1, about 10:1, or about 20:1). In certain embodiments, the PDI is less than about 0.3 when measured at a loading ratio of about 1:1 to about 30:1 (e.g., about 5:1, about 10:1, or about 20:1).

In certain embodiments, the block copolymer has a Zeta potential (mV) of about 30 to about 65. For example, the Zeta potential (mV) can be about 30 to about 60, about 35 to about 60, or about 40 to about 60. The Zeta potential can be measured as described in the Examples herein, for example using electrophoretic light scattering.

In some embodiments, the nanoparticle comprises a barcode construct covalently attached to the nanoparticle, as described in U.S. Patent Application Publication No. 2022/033309, the entirety of which is hereby incorporated by reference. As described in U.S. Patent Application Publication No. 2022/033309, the presence of the barcode construct can allow for identification of PNPs of interest, for example by determining the presence of a PNP in particular tissue or by performance in an assay. It should be understood that although certain examples in this application are performed on PNPs that include the barcode, similar performance would be expected by PNPs that lack the barcode. polymer nanoparticle.

In one illustrative aspect, the polymer nanoparticles may be used as delivery vehicles according to the present disclosure. In some embodiments, the non-viral delivery vehicle comprises one or more nanoparticle forming polymers. In some embodiments, the non-viral delivery vehicle comprises polymer nanoparticles. In some embodiments, the non-viral delivery vehicle is not a lipid based system. In some embodiments, the non-viral delivery vehicle comprises polymer nanoparticles made from controlled living/radical polymerization processes. It will be appreciated that the identity of the monomer units is not particularly limited so long as the monomer units being used are compatible with a controlled living/radical polymerization, such as reversible-deactivation radical polymerization, atom transfer radical polymerization (ATRP), reversible addition fragmentation chain transfer polymerization (RAFT), iodine-transfer polymerization (ITP), selenium-centered radical-mediated polymerization, telluride-mediated polymerization (TERP), stibine-mediated polymerization, ring-opening polymerization, or like polymerization processes. In some embodiments, the polymer nanoparticles may be made by RAFT copolymerization to synthesize a diverse set of block copolymers, and to screen their ability to form complexes with a payload. In one aspect, polymer nanoparticles (e.g., RAFT copolymers) may be produced by chemically bonding a payload to a constituent polymer, such as by the grafting of the payload onto RAFT copolymers using chain transfer agents, and subsequently assembling the polymers into a delivery vehicle.

In some embodiments, a composition comprises a polymer nanoparticle as described herein and a nucleic acid (sometimes called a payload) complexed to the polymer nanoparticle. For example, the nucleic acid may be complexed to the nanoparticle through electrostatic interactions. In certain preferred embodiments, the polymer nanoparticle of the composition serves as a transfection agent to deliver a nucleic acid to a cell.

In certain embodiments, the nucleic acid is an RNA (e.g., an mRNA, a microRNA, a circular RNA, antisense oligonucleotides, or an siRNA) or a DNA (e.g., an ssDNA, a dsDNA, or a complimentary coding DNA (cDNA), or a DNA plasmid). In certain embodiments, the nucleic acid is an mRNA. In certain embodiments, the nucleic acid is a circular RNA (circRNA). In certain embodiments, the nucleic acid is a DNA plasmid (pDNA).

In certain embodiments, the ratio of nanoparticle to nucleic acid is about 1:1 to about 30:1 by weight, such as about 1:1 to about 20:1 by weight. For example, the ratio of nanoparticle to nucleic acid may be about 2:1 to about 30:1, about 5:1 to about 30:1, about 10:1 to about 30:1, or about 20:1 to about 30:1 by weight. In some embodiments, the ratio of nanoparticle to nucleic acid is less than about 30:1, less than about 25:1, less than about 20:1, less than about 15:1, less than about 10:1, less than about 7:1, or less than about 5:1 by weight. In some embodiments, the ratio of nanoparticle to nucleic acid is about 5:1 to about 10:1 by weight. In some embodiments, the ratio of nanoparticle to nucleic acid is about 6:1 to about 10:1 by weight.

In certain embodiments, the payload (e.g., a nucleic acid, such as a circRNA, mRNA, or pDNA, complexed to the polymer nanoparticle) may be present in the composition in an amount of about 150 ng to about 1000 ng, such as about 200 ng to about 1000 ng, about 250 ng to about 1000 ng, about 300 ng to about 1000 ng, about 400 ng to about 1000 ng, about 500 ng to about 1000 ng, about 600 ng to about 1000 ng, or about 750 ng to about 1000 ng. In certain embodiments, the payload may be present in the composition in an amount of at least about 150 ng, at least about 200 ng, at least about 250 ng, at least about 300 ng, at least about 400 ng, at least about 500 ng, at least about 600 ng, or at least about 750 ng.

In certain embodiments, the polymer nanoparticle may be present in the composition in an amount of about 0.001 mg/mL to about 0.05 mg/mL, such as about 0.001 mg/mL to about 0.03 mg/mL. In certain embodiments, the polymer nanoparticle may be present in the composition in an amount of less than about 0.05 mg/mL, less than about 0.04 mg/mL, or less than about 0.03 mg/mL.

The polymer nanoparticles described herein are capable of interacting with (e.g., encapsulating or complexing with) nucleic acids. In some embodiments, the encapsulation efficiency is greater than about 70%, greater than about 75%, greater than about 80%, greater than about 85%, greater than about 90%, greater than about 95%, or greater than about 97%. In certain embodiments, the encapsulation efficiency is about 90% to about 99% and preferably is about 95% to about 99%.

In certain embodiments, the transfection efficiency is least about 10% into cells, at least about 30%, at least about 50%, or at least about 70% into cells, for example mammalian cells such as neuronal cells. In certain embodiments, the transfection efficiency is about 10% to about 99%, about 30% to about 99%, about 50% to about 99%, about 60% to about 99%, about 70% to about 99%, about 80% to about 98%, about 85% to about 98%, about 90% to about 99%, or about 95% to about 99%. In certain embodiments, the transfection efficiency is least about 10% into cells, for example mammalian cells such as neuronal cells. In certain embodiments, the transfection efficiency is about 10% to about 45%, about 10% to about 40%, about 10% to about 35%, about 15% to about 35%, or about 15% to about 30%. In certain embodiments, the transfection efficiency is about 10% to about 99%. In certain embodiments, the transfection efficiency is about 50% to about 99%. In certain embodiments, the transfection efficiency is about 85% to about 99%.

In certain embodiments, the transfection efficiency is about 85% to about 99% and the ratio of nanoparticle to nucleic acid is about 1:1 to about 30:1 by weight, such as about 1:1 to about 20:1. For example, the transfection efficiency may be about 85% to about 99% and the ratio of nanoparticle to nucleic acid may be about 2:1 to about 30:1, about 5:1 to about 30:1, about 10:1 to about 30:1, or about 20:1 to about 30:1 by weight. In certain embodiments, the transfection efficiency is about 85% to about 99% and the ratio of nanoparticle to nucleic acid is about 1:1, about 2:1, about 3:1, about 4:1, about 5:1, about 6:1, about 7:1, about 10:1, about 15:1, about 20:1, about 25:1, or about 30:1 by weight. In certain embodiments, the transfection efficiency is about 85% to about 99% and the ratio of nanoparticle to nucleic acid is about 1:1 to about 10:1 by weight. In certain embodiments, the transfection efficiency is about 85% to about 99% and the ratio of nanoparticle to nucleic acid is about 5:1 to about 10:1 by weight. In certain embodiments, the transfection efficiency is about 85% to about 99% and the ratio of nanoparticle to nucleic acid is about 6:1 to about 10:1 by weight.

In some embodiments, the nucleic acid (e.g., a mRNA, a circRNA, or a pDNA) is less than about 5 kb. In some embodiments, is about 500 bp to about 2000 bp, about 50 bp to about 1000 bp, about 250 to about 1000 bp, or about 500 bp to about 1000 bp.

In certain embodiments, the polymers of the present disclosure are capable of delivering a payload (e.g., a nucleic acid such as an mRNA a circRNA, or a pDNA) to neuronal cells to cause a change in protein expression. In some embodiments, the mRNA corresponds to a protein that is capable of being expressed in the neuronal cells (e.g., in the brain). In certain embodiments, the compositions (e.g., PNP and nucleic acid) are administered to a mammal, such as a mouse, via an injection, such as a intracerebroventricular injection.

In one embodiment, a composition comprising a polymer nanoparticle (e.g., a polymer nanoparticle derived from a controlled living/radical polymerization process, such as RAFT polymer) associated with a nucleic acid construct is provided. In another embodiment, a method of in vivo screening to identify a desired polymer nanoparticle (e.g., a polymer nanoparticle derived from a controlled living/radical polymerization process, such as RAFT polymer) associated with a nucleic acid construct for use as a delivery vehicle is provided. In another embodiment, a method of treating a patient with a disease is provided comprising administering to the patient the polymer nanoparticle identified in the screening method.

In some embodiments, the method of in vivo screening for a desired polymer nanoparticle for use as a delivery vehicle comprises, (a) preparing a library comprising two or more types of polymer nanoparticles, wherein each polymer nanoparticle is associated with a nucleic acid construct comprising a different polynucleotide barcode, (b) administering the library to an animal, (c) removing cells or tissues from the animal, (d) isolating the nucleic acid constructs from the cells or tissues of the animal, (e) detecting the nucleic acid constructs in the cells or tissue of the animal, and (f) identifying the desired polymer nanoparticle for use as a delivery vehicle. In various embodiments, the nucleic acid construct can be detected by, for example, the polymerase chain reaction (PCR), isothermal amplification, or sequencing the nucleic acids in the cells or tissues of the animal.

The compositions described herein can be used for treating disease. For example, the polymer nanoparticles may be able to deliver a nucleic acid (i.e., a payload) that provides a therapeutic benefit to a patient. In certain embodiments, the polymer nanoparticle is able to deliver the nucleic acid to cells, such as neuronal cells. The compositions described herein can be used for treating disease, including neurological diseases (e.g., Parkinson's Disease or Alzheimer's Disease). In certain embodiments, the polymer nanoparticle delivers the nucleic acid to the brain or the cells of the central nervous system, for example microglia, astrocytes, oligodendrocytes, and neurons.

In some embodiments, a method of treating a patient with a disease is provided, comprising administering to the patient the polymer nanoparticle, wherein the polymer nanoparticle further comprises a drug payload, such as a nucleic acid (e.g., polynucleotide) or a protein payload, or a small molecule therapeutic or luminescent molecule payload, and treating the disease in the patient. In another embodiment, a method of treating a patient with a disease is provided comprising administering to the patient the composition of the present disclosure. In some embodiments, the polymer nanoparticles are delivered to the brain of the patient.

In some embodiments, a method of transfecting a cell, such as a mammalian cell (e.g., a neuronal cell) is provided comprising contacting the cell with an effective amount of a composition according to the present disclosure. In some embodiments, the method is in vitro or in vivo.

In some embodiments, the polymer nanoparticles have low toxicity to cells.

In various embodiments, any suitable route for administration of the polymer nanoparticles associated with nucleic acid constructs, or for the method of treatment can be used including parenteral administration or oral administration. Suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. In one embodiment, means for parenteral administration include needle (including microneedle) injectors, needle-free injectors and infusion techniques. In other embodiments, oral or pulmonary routes of administration can be used.

It will be appreciated that RAFT polymerization is generally known in the art. Suitable reagents, monomers, and conditions for RAFT polymerization previously investigated can be used in the copolymers, methods, and compositions described herein, such as those described in U.S. Pat. Nos. 9,006,193, 9,464,300, and 9,476,063, the disclosures of each of which are incorporated by reference in their entirety.

Chain transfer agents (CTAs) useful in connection with the present disclosure are known in the art. The identity of the CTA is not particularly limited. It will be appreciated that chain transfers steps that form the basis of RAFT polymerization involve a reversible transfer of a functional chain end-group (typically a thiocarbonylthio group, Z—C(═S)S—R) between chains and the propagating radicals. The overall process is comprised of the insertion of monomers between the R— and Z—C(═S)S-groups of a RAFT agent (CTA), which form the a and @ end-group of the majority of the resulting polymeric chains. Suitable CTAs for use in connection with the present disclosure include but are not limited to trithiocarbonates (Z=S-alkyl), dithiobenzoates (Z=Ph), dithiocarbamate (Z=N-alkyl), xanthates (Z=O-alkyl), and the like. (See, Sébastien Perrier, Macromolecules 2017 50 (19), 7433-7447). In some embodiments, RAFT copolymerization may be achieved using chain transfer agents (CTAs) containing one or more terminal carboxyl groups in order to obtain carboxy terminated polymers with ends available for bonding to the payload via the methods described above. In this embodiment, when the resulting mono or di-carboxy terminated polymer is dispersed in a low pH (e.g., a pH of less than 6) buffer, both ends of the polymer are exposed and available for labeling via EDC/NHS chemistry. In this embodiment, when the polymer is transferred to a physiological pH (~pH 7), the core blocks self-assemble, encapsulating the payload in the hydrophobic core, to be released and exposed upon acidification in the endosomal compartment of a cell. In some embodiments, the first or second chain transfer agent can be selected from the group consisting of bis(carboxymethyl)trithiocarbonate, bis(2-amino-2-oxoethyl) trithiocarbonate, bis[4-(2-hydroxyethoxycarbonyl)benzyl] trithiocarbonate, 4-cyano-4-(ethylsulfanylthiocarbonyl) sulfanylvpentanoic acid, 4-cyano-4-((phenylcarbonothioyl)thio)pentanoic acid, and 4-cyano-4-[(dodecylsulfanylthiocarbonyl)sulfanyl] pentanoic acid, 4-cyano-4-(thiobenzoylthio)pentanoic acid, 2-cyano-2-propyl benzodithioate, cyanomethyl methyl(phenyl)carbamodithioate, 2-cyano-2-propyl dodecyl trithiocarbonate, 2-(dodecylthiocarbonothioylthio)-2-methylpropionic acid, cyanomethyl dodecyl trithiocarbonate, 2-cyano-2-propyl 4-cyanobenzodithioate, and the like.

In another illustrative embodiment, the polymer nanoparticle composition can be coated with one or more polymers to protect the compositions from immune responses or to enhance endosomal escape. In one embodiment, the one or more polymers used for coating comprise polyethylene glycol. In another embodiment, the one or more polymers used for coating comprise polyethylene glycol poly-L-lysine. In yet another embodiment, the one or more polymers used for coating comprise polyethylenimine. In an additional embodiment, the one or more polymers used for coating comprise polyethylene glycol poly-L-lysine and polyethylenimine.

In some embodiments, a single chain transfer agent can be used in the RAFT polymerization process in connection with the present disclosure. In some embodiments, for a block polymer having more than one block, one or more single chain transfer agents can be used in the RAFT polymerization process in connection with the present disclosure. In some embodiments, for a block polymer having two blocks, a first chain transfer agent and a second chain transfer agent (which can be the same or different) can be used at each step of the RAFT polymerization process in connection with the present disclosure. In some embodiments, for a block polymer having three blocks, a first chain transfer agent, a second chain transfer agent, and a third chain transfer agent (which can be the same or different) can be used at each step of the RAFT polymerization process in connection with the present disclosure.

It will be appreciated that a variety of solvents can be used in the RAFT polymerization method steps and purification steps described herein. Suitable solvents include, but are not limited to, 2-Chloroethanol, Acetic Acid (Glacial), Acetone, Acetonitrile, Acetophenone, Aniline, Benzaldehyde, Benzyl Acetate, Carbon disulfide, Cyclohexane, Cyclohexanol, Di(ethylene glycol), Di(propylene glycol), Diacetone alcohol, Diethyl ether, Dimethylsulfoxide, Ethanol, Ethyl acetate, Ethylene glycol, Formaldehyde (37% solution), Formamide, Formic acid, Formic acid (96%), Hexane, Isobutanol, Isopropanol, Isopropyl acetate, Isopropyl ether, m-Cresol, Methanol, Methyl acetate, Methyl ethyl ketone, Mineral Oil, N,N-Dimethylformamide, n-Butanol, n-Octane, n-Propanol, Propylene glycol, Pyridine, Sodium Acetate Buffer, t-Butanol, Tetrahydrofuran, Trifluoroacetic acid, water, and the like, and combinations thereof.

### Alternative Embodiments

The following clauses, and combinations thereof, provide various additional illustrative aspects of the disclosure described herein. The various embodiments described in any other section of this patent application, including the section titled “DETAILED DESCRIPTION,” “REPRESENTATIVE EMBODIMENTS,” and the “EXAMPLES” are applicable to any of the following embodiments described in the clauses enumerated below.
1. A block copolymer comprising
   a first block comprising a homopolymer of monomer units represented by formula I:

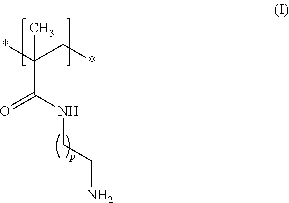

or a salt thereof, wherein:
   p is 0 or an integer selected from 1-3,
   wherein the * represent the connecting points to the rest of the block copolymer; and
a second block comprising a homopolymer of monomer units represented by formula II:

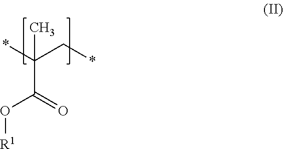

or a salt thereof, wherein:
   R¹ is alkyl; and
   wherein each * represents a connecting point to the rest of the block copolymer.
2. The block copolymer of clause 1, wherein p is 1.
3. The block copolymer of any one of the preceding clauses, wherein R¹ is C₁-C₆ alkyl.
4. The block copolymer of any one of the preceding clauses, wherein R¹ is butyl.
5. The block copolymer of any one of clauses 1-3, wherein R¹ is methyl.
6. The block copolymer of any one of the preceding clauses, wherein R¹ is unsubstituted.
7. A block copolymer comprising a first block and a second block, wherein:
   the first block comprises a homopolymer of poly (2-aminoethyl methacrylamide) p(AEMA); and
   the second block comprises a homopolymer of poly (butylmethacrylate) p(BMA) or a homopolymer of poly(methylmethacrylate) p(MMA).
8. The block copolymer of any one of the preceding clauses, wherein the copolymer has
   (i) an overall molecular weight (M_{w}) of about 25 kDa to about 60 kDa,
   (ii) a theoretical corona to core ratio (CCR) of at least about 5, or
   (iii) both (i) and (ii).
9. The block copolymer of any one of the preceding clauses, wherein the copolymer has an overall molecular weight (M_{w}) of about 30 kDa to about 60 kDa.
10. The block copolymer of any one of the preceding clauses, wherein the theoretical CCR is about 5 to about 40 (e.g., about 7 to about 25).
11. The block copolymer of any one of the preceding clauses, wherein the block copolymer has a Zeta potential (mV) of about 30 to about 65.
12. A polymer nanoparticle comprising a block copolymer according to any one of the preceding clauses.
13. The polymer nanoparticle of clause 12, wherein the nanoparticle has a PDI of about 0.05 to about 0.6, as measured by DLS.
14. The polymer nanoparticle of clause 12, wherein the nanoparticle has a PDI of about 0.05 to about 0.45, as measured by DLS.
15. The polymer nanoparticle of clause 12, wherein the nanoparticle has a PDI of about 0.05 to about 0.3, as measured by DLS.
16. A composition comprising:
   a polymer nanoparticle according to any one of clauses 12-15, and
   a nucleic acid complexed to the polymer nanoparticle.
17. The composition of clause 16, wherein the nucleic acid is RNA.
18. The composition of clause 16 or 17, wherein the nucleic acid is a circular RNA or mRNA.
19. The composition according to any one of clauses 16-18, wherein the nucleic acid complexed to the polymer nanoparticle transfects a cell.
20. The composition according to any one of clauses 16-19, wherein the composition has a cell transfection efficiency of about 10% to about 45%.
21. The composition according to clause 19 or 20, wherein the cell is a mammalian cell.
22. The composition according to any one of clauses 19-21, wherein the cell is a neuronal cell.
23. A method of treating a disease in a patient in need thereof, the method comprising administering a composition according to any one of clauses 16-20.
24. The method of clause 23, wherein the polymer nanoparticles are delivered to the brain of the patient.

### Additional Alternative Embodiments

The following clauses, and combinations thereof, provide various additional illustrative aspects of the disclosure described herein. The various embodiments described in any other section of this patent application, including the section titled “DETAILED DESCRIPTION,” “REPRESENTATIVE EMBODIMENTS,” and the “EXAMPLES” are applicable to any of the following embodiments described in the embodiments enumerated below.
1. A block copolymer comprising
   a first block comprising a first homopolymer of monomer units of formula I:

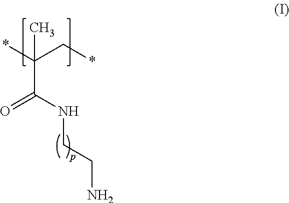

or a salt thereof, wherein:
   p is 0 or an integer selected from 1-3,
   wherein the * represent the connecting points to the rest of the block copolymer; and
a second block comprising a second homopolymer of monomer units of formula II:

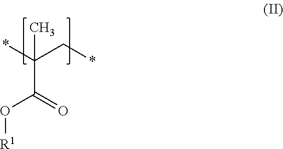

or a salt thereof, wherein:
   R¹ is alkyl; and
   wherein each * represents a connecting point to the rest of the block copolymer,
wherein the first block has a molecular weight (Mₙ) of at least about 15 kDa, and
wherein the block copolymer has a ratio of monomer units of formula I to monomer units of formula II of about 80:20 to about 99:1.
2. The block copolymer of embodiment 1, wherein p is 1.
3. The block copolymer of any one of the preceding embodiments, wherein R¹ is C₁-C₆ alkyl.
4. The block copolymer of any one of the preceding embodiments, wherein R¹ is butyl.
5. The block copolymer of any one of embodiments 1-3, wherein R¹ is methyl.
6. The block copolymer of any one of the preceding embodiments, wherein R¹ is unsubstituted.
7. A block copolymer comprising a first block and a second block, wherein:
   the first block comprises a first homopolymer of poly (2-aminoethyl methacrylamide) p(AEMA); and
   the second block comprises a second homopolymer of poly (butylmethacrylate) p(BMA) or a homopolymer of poly(methylmethacrylate) p(MMA)
   wherein the first block has a molecular weight (Mₙ) of at least about 15 kDa, and
   wherein the block copolymer has a ratio of monomer units of formula I to monomer units of formula II of about 80:20 to about 99:1.
8. The block copolymer of any one of the preceding embodiments, wherein the block copolymer has
   (i) an overall molecular weight (M_{w}) of about 25 kDa to about 70 kDa,
   (ii) a theoretical corona-to-core ratio (CCR) of at least about 5, or
   (iii) both (i) and (ii).
9. The block copolymer of any one of the preceding embodiments, wherein the block copolymer has an overall molecular weight (M_{w}) of about 25 kDa to about 60 kDa.
10. The block copolymer of any one of the preceding embodiments, wherein the block copolymer has an overall molecular weight (M_{w}) of about 30 kDa to about 70 kDa.
11. The block copolymer of any one of the preceding embodiments, wherein the block copolymer has an overall molecular weight (M_{w}) of about 30 kDa to about 60 kDa.
12. The block copolymer of any one of embodiments 1 to 7, wherein the block copolymer has
   (i) an overall molecular weight (Mₙ) of about 15 kDa to about 50 kDa,
   (ii) a theoretical corona-to-core ratio (CCR) of at least about 5, or
   (iii) both (i) and (ii).
13. The block copolymer of any one of the preceding embodiments, wherein the block copolymer has an overall molecular weight (Mₙ) of about 15 kDa to about 25 kDa.
14. The block copolymer of any one of the preceding embodiments, wherein the theoretical CCR is about 5 to about 40 (e.g., about 7 to about 25).
15. The block copolymer of any one of the preceding embodiments, wherein the block copolymer has a Zeta potential (mV) of about 30 to about 65.
16. A polymer nanoparticle comprising a block copolymer according to any one of the preceding embodiments.
17. The polymer nanoparticle of embodiment 16, wherein the nanoparticle has a PDI of about 0.05 to about 0.6, as measured by DLS.
18. The polymer nanoparticle of embodiment 16, wherein the nanoparticle has a PDI of about 0.05 to about 0.45, as measured by DLS.
19. The polymer nanoparticle of embodiment 16, wherein the nanoparticle has a PDI of about 0.05 to about 0.3, as measured by DLS.
20. A composition comprising:
   a polymer nanoparticle according to any one of embodiments 16-19, and
   a nucleic acid complexed to the polymer nanoparticle.
21. The composition of embodiment 20, wherein the nucleic acid is RNA.
22. The composition of embodiment 20 or 21, wherein the nucleic acid is a circular RNA or mRNA.
23. The composition according to any one of embodiments 20-22, wherein the nucleic acid complexed to the polymer nanoparticle transfects a cell.
24. The composition according to any one of embodiments 20-23, wherein the composition has a cell transfection efficiency of about 10% to about 45%.
25. The composition according to any one of embodiments 20-23, wherein the composition has a cell transfection efficiency of about 50% to about 99%.
26. The composition according to any one of embodiments 20-23, wherein the composition has a cell transfection efficiency of about 85% to about 99%.
27. The composition according to any one of embodiments 23-26, wherein the cell is a mammalian cell.
28. The composition according to any one of embodiments 23-27, wherein the cell is a neuronal cell.
29. A method of treating a disease in a patient in need thereof, the method comprising administering a composition according to any one of embodiments 20-28.
30. The method of embodiment 29, wherein the polymer nanoparticles are delivered to the brain of the patient.

### EXAMPLES

### Polymer Nanoparticle Synthesis

Some examples in this disclosure relate to a p(AEMA)-b-p(BMA) polymer nanoparticle (PNP) composition that has successfully delivered RNA to the brain in mice upon local administration. This invention works by leveraging a p(AEMA)-b-p(BMA) PNP design that includes a Block 1 TheoreticalDegree of Polymerization (DP)=160 or 180, Block 2 Theoretical DP=20, and Chain Transfer Agent (CTA)-to-Initiator (CTA:I) ratio=1 to reproducibly create a cationic PNP capable of transfecting cells in the brain of mice. This polymer was synthesized using the following reaction conditions:

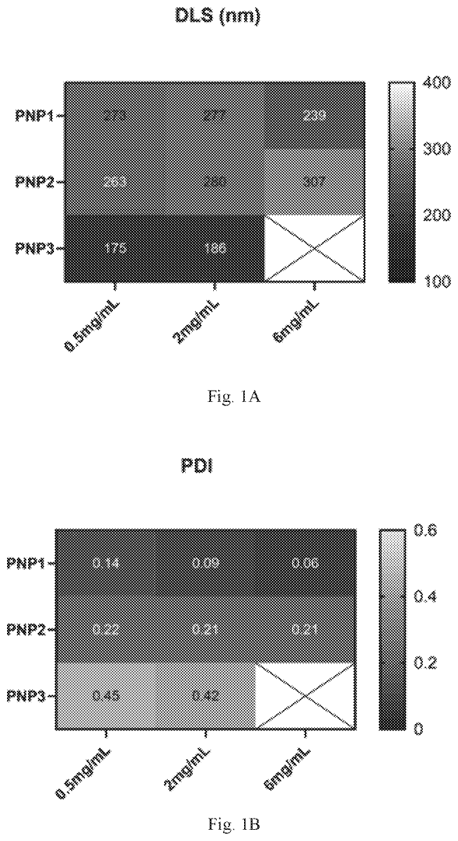

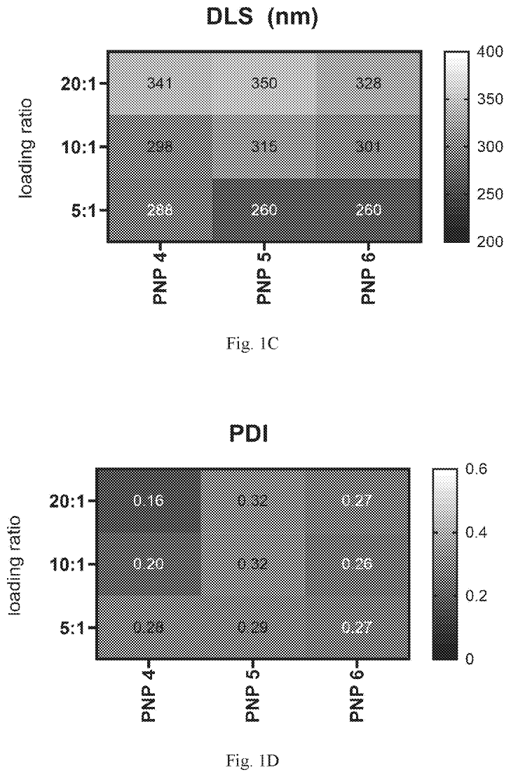

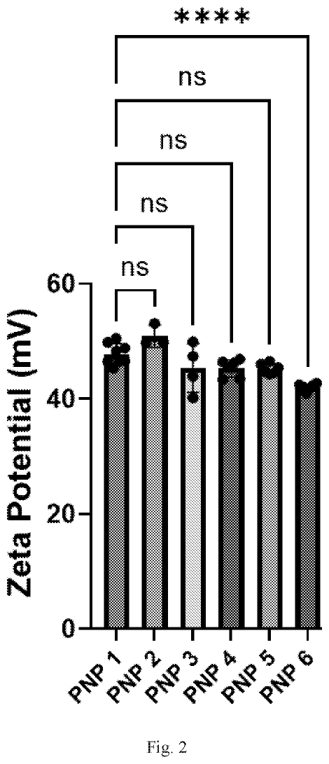

Reacted at 70° C. for 24 hours in 3M Sodium Acetate Buffer, and then was purified using 4 mL 3K MWCO Amicon filters and Milli-Q water adjusted to pH 3.5 to remove residual salt and reagent impurities. The purified solution was frozen for at least 12 hours in −80° C. conditions before being lyophilized for at least 72 hours. Two different PNPs were produced by the same reaction conditions at two different times using two separate sets of reagents, yielding “PNP 1” and “PNP 2”. Three additional PNPs 4-6 were produced by the same reaction conditions at different times using separate sets of reagents. These polymers were then formulated in aqueous conditions using 1×PBS, which yielded characteristics documented in FIGS. 1A-1B and 2, or formulated in aqueous conditions using 5% glucose solution and loaded with circRNA, which yielded characteristics documented in FIGS. 1C-1D.

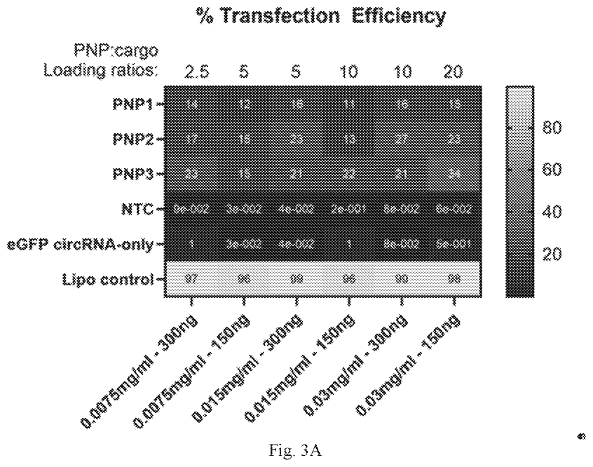

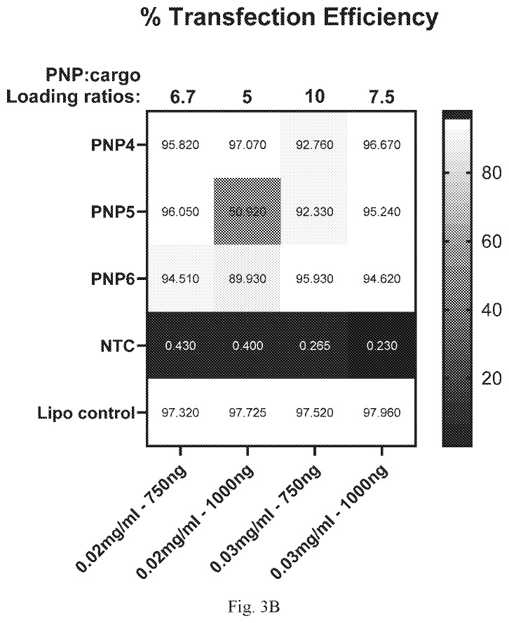

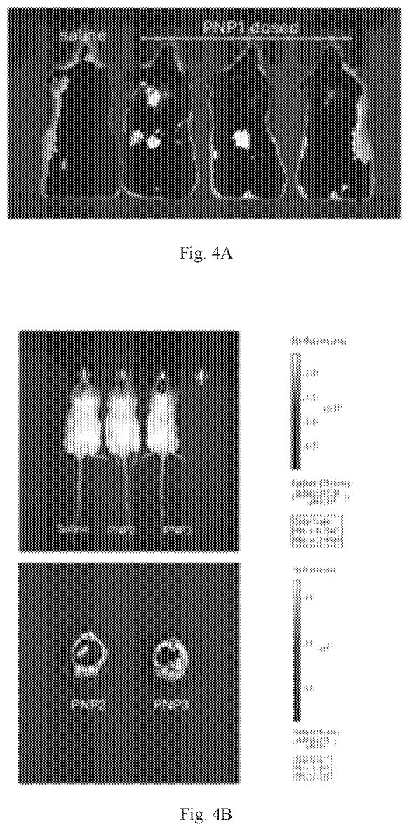

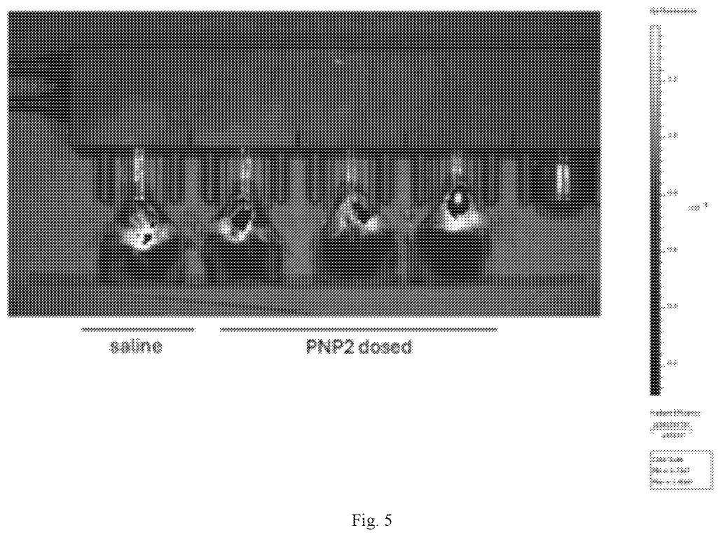

Next the PNP replicates were tested for in vitro delivery of eGFP circRNA in HEK293T cells, showing approximately 20% transfection efficiency for PNPs 1-3 (FIG. 3A), and approximately 90-95% transfection efficiency for PNPs 4-6 (FIG. 3B). They were then used for in vivo intracerebroventricular (ICV) delivery of mCherry circRNA to the brain of wild-type mice (FIGS. 4A and 4B) and ICV delivery of Cre mRNA to the brains of Ai9 mice for PNP 2 (FIG. 5).

This same composition was synthesized again (PNP 3) as described above except for using a Block 1 TheoreticalDegree of Polymerization (DP) value of 180. Though slightly different in some physical characteristics (FIGS. 1A and 1B), this PNP also successfully delivered payload in vivo (FIG. 4B).

Some examples in this disclosure relate to p(AEMA)-b-p(BMA) compositions that may have a lowering the PDI, which is an indicator of monodispersity (i.e., uniformity), as measured using DLS, and/or a lower PDI values when tested at higher PNP concentration (e.g., 6 mg/mL) than previous PNPs, having desired zeta potential measurement data. The coefficient of variation (% CV) values for the zeta potential measurements for PNP 1 and PNP 2 were both <3.8%, and the % CV value for PNP 3 was <9.3% indicating successful consistency in a critical quality attribute of % CV values<10%. The disclosed PNPs may also demonstrate solubility of the PNP both alone in solution as well as when loaded with nucleic acid payloads.

Diblock copolymers were synthesized using reversible addition-fragmentation chain transfer (RAFT) polymerization with reagents and amounts listed in Table 2. Block 1 reagents, including 2-aminoethylmethylacrylamide (AEMA) as the monomer, 4-Cyano-4-[(ethylsulfanylthiocarbonyl) sulfanyl]pentanoic acid (ECT) as the chain transfer agent (CTA), 4,4′-Azobis(4-cyanovaleric acid) (ACVA) as the initiator, and 3M sodium acetate buffer as the solvent, were combined in a 100 mL round-bottom flasks, purged with argon, and heated to 70° C. for 24 hours. The reaction products were purified using 4 mL 3K MWCO Amicon filters and Milli-Q water adjusted to pH 3.5 to remove residual salt and reagent impurities. Finally, the purified materials were frozen for at least 12 hours at −80° C. and then lyophilized for 72 hours.

Block copolymers were prepared using Reversible Addition-Fragmentation Chain Transfer (RAFT) polymerization, using the monomers shown in Table 1 below. Block copolymers included:
p(AEMA)-b-p(BMA);
   with block 1 degree of polymerization ranging from about 160 to about 180, and block 2 degree of polymerization ranging of about 20. The corresponding Mₙ-calculated CCR values for PNPs 1, 4, 5, and 6 are all approximately 8.5-9.5.

TABLE 1
Monomers used in the nanoparticle forming polymers (PNPs).

| Monomer Name | Abbreviation | CAS | Structure |
|---|---|---|---|
| Butyl methacrylate | BMA | 97-88-1 | 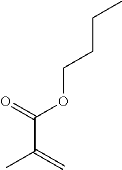 |
| 2-Aminoethyl methacrylamide | AEMA | 76259-32-0 | 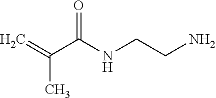 |

TABLE 2
PNP composition summary

| | | | | | Overall | Overall | % AEMA | % BMA |
|---|---|---|---|---|---|---|---|---|
| | B1 AEMA | B2 BMA | mCTA | mCTA | Diblock | Diblock | Monomer in | Monomer |
| | Theoretical | Theoretical | Mw | Mn | Copolymer | Copolymer | Diblock | in Diblock |
| PNP | DP | DP | (kDa) | (kDa) | Mw (kDa) | Mn (kDa) | Copolymer | Copolymer |
| 1 | 160 | 20 | 24.2 | 18.2 | 66.1 | 20.3 | 98.2% | 1.8% |
| 2 | 160 | 20 | 27.3 | 17.4 | 36.6 | | 98.5% | 1.5% |
| 3 | 180 | 20 | 39.1 | 21.6 | 50.6 | | 98.9% | 1.1% |
| 4 | 160 | 20 | | 19.0 | | 21.2 | 98.0% | 2.0% |
| 5 | 160 | 20 | | 19.0 | | 21.2 | 97.4% | 2.6% |
| 6 | 160 | 20 | | 19.0 | | 21.0 | 97.7% | 2.3% |

### Procedure for Particle Size Measurement Using Dynamic Light Scattering (DLS):

Di-block polymers in a dry solid state were rehydrated using ultrapure water or suitable aqueous buffer to a concentration in the range of about 1-40 mg/mL, utilizing a combination of agitation by orbital shaker and sonication to disperse the polymer into nanoparticles. Solutions were centrifuged for 5 minutes to settle any dust or large aggregates present. Aliquots were transferred to a flat-bottom well plate suitable for measurement and diluted tenfold, resulting in a polymer concentration in the range of about 0.1-6 mg/mL. The plate was measured using a Wyatt DynaPro Plate Reader III utilizing 7 acquisitions per well. Suitable data filters were applied to the resulting autocorrelation functions to remove low-quality acquisitions. Representative data filter parameters include a baseline acceptance criterion of 1±0.05, a minimum amplitude of 0.05, and a maximum sum-of-squares error of 100. The Z-average diameter was used to provide the hydrodynamic diameter. Results are shown in FIGS. 1A and 1B for PNPs 1-3. Results are shown in FIGS. 1C and 1D for PNPs 4-6 loaded via complexation with a circular RNA (circRNA) expressing the eGFP fluorescent reporter protein.

### Procedure for Zeta Potential Measurement Using Electrophoretic Light Scattering:

Approximately 4-8 mg of polymer was dissolved in Milli-Q water to a concentration of 2 mg/mL in an Eppendorf tube. Samples were agitated by vortexing and sonication until completely dissolved before being left to stand at room temperature overnight. The next day, samples were transferred into a Malvern p1070 capillary cell, and the zeta potential of each sample was measured using the electrophoretic light scattering capability of a Malvern Zetasizer Nano ZS. An average of 5 measurement scans was used to provide the zeta potential value. Results are shown in FIG. 2.

### Procedure for In Vitro Transfection Efficiency:

To measure the in vitro transfection efficiency and viability upon transfection, the PNPs were loaded with a circular RNA (circRNA) expressing the fluorescent eGFP reporter protein. These were dosed into HEK293T cells. Cells were seeded in 96 well plates at 30,000 cells per well or in 48 well plates at 80,000 cells per well, and incubated for 24 hours before treating with 6 different doses of PNP and circRNA cargo:

| mg/mL PNP | ng circRNA | PNP:circRNA ratio |
|---|---|---|
| 0.0075 | 150 | 5 |
| 0.015 | 150 | 10 |
| 0.03 | 150 | 20 |
| 0.0075 | 300 | 2.5 |
| 0.015 | 300 | 5 |
| 0.03 | 300 | 10 |
| 0.02 | 750 | 6.7 |
| 0.02 | 1000 | 5 |
| 0.03 | 750 | 10 |
| 0.03 | 1000 | 7.5 |

The number of GFP-expressing cells were counted via flow cytometry at 48 hours post-dosing. Transfection efficiency was calculated as defined by the equation below. Results are shown for PNP IDs 1-3 in FIG. 3A, and for PNP IDs 4-6 in FIG. 3B.

$$Transfection efficiency ( \% ) = \frac{GFP expressing Cells}{Total Cells}$$

Viability data was also captured by staining cells with zombie violet, which specifically only stains dead cells. Viability % was calculated as defined by the equation below.

$$Cell Viability ( \% ) = \frac{non - Violet cells}{Total Cells}$$

### Ribogreen Assay for Calculating Encapsulation Efficiency

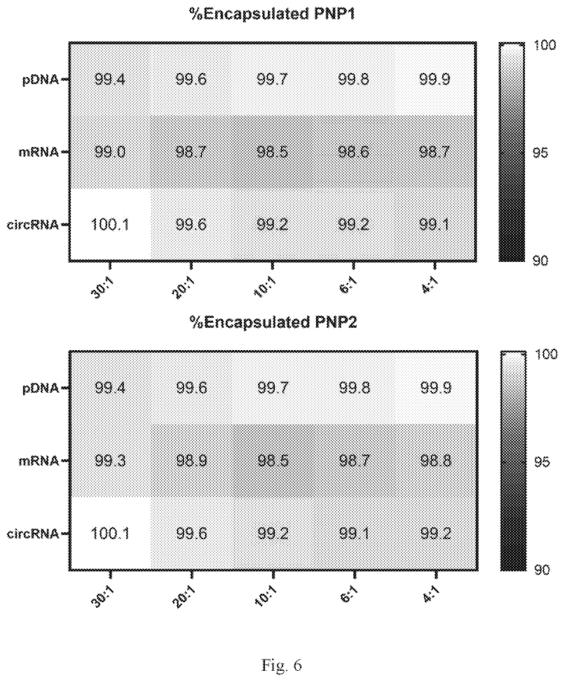

Polymers loaded at 5 different PNP:cargo weight ratios (30:1, 20:1, 10:1, 6:1, and 4:1) at a 0.5 mg/mL PNP concentration for 3 payloads: eGFP circRNA, eGFP mRNA, and eGFP-expressing pDNA. After 30 minutes of incubation 100 μL of Ribogreen™ Reagent (1:1000 dilution) is added to the samples for a total volume of 200 μL. The samples are scanned at ex. 485/em. 530 for fluorescence of the DNA intercalating dye. The fluorescence intensity is correlated to a standard curve of each cargo to calculate the encapsulation efficiency. Cargo that has been shielded by the PNP will not fluoresce leading to lower fluorescence intensity for PNPs with greater encapsulation efficiency. The results of all 5 ratios are shown in FIG. 6.

1. A block copolymer comprising
a first block comprising a first homopolymer of monomer units of formula I: 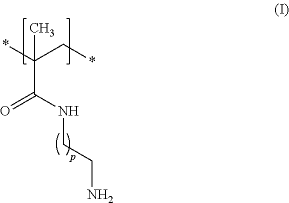
or a salt thereof, wherein:
p is 0 or an integer selected from 1-3,
wherein the * represent the connecting points to the rest of the block copolymer; and
a second block comprising a second homopolymer of monomer units of formula II: 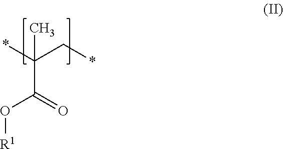
or a salt thereof, wherein:
R¹ is alkyl; and
wherein each * represents a connecting point to the rest of the block copolymer,
wherein the first block has a molecular weight (Mₙ) of at least about 15 kDa, and
wherein the block copolymer has a ratio of monomer units of formula I to monomer units of formula II of about 80:20 to about 99:1.

2. The block copolymer of claim 1, wherein p is 1.

3. The block copolymer of claim 1, wherein R¹ is C₁-C₆ alkyl.

4. The block copolymer of claim 3, wherein R¹ is butyl.

5. The block copolymer of claim 3, wherein R¹ is methyl.

6. The block copolymer of claim 1, wherein:
the first homopolymer is poly (2-aminoethyl methacrylamide) p(AEMA); and
the second homopolymer is poly (butylmethacrylate) p(BMA) or poly (methylmethacrylate) p(MMA).

7. The block copolymer of claim 1, wherein the block copolymer has
(i) an overall molecular weight (M_{w}) of about 25 kDa to about 70 kDa,
(ii) a theoretical corona-to-core ratio (CCR) of at least about 5, or
(iii) both (i) and (ii).

8. The block copolymer of claim 1, wherein the block copolymer has
(i) an overall molecular weight (Mₙ) of about 15 kDa to about 50 kDa,
(ii) a theoretical corona-to-core ratio (CCR) of at least about 5, or
(iii) both (i) and (ii).

9. The block copolymer of claim 1, wherein the block copolymer has an overall molecular weight (Mₙ) of about 15 kDa to about 25 kDa.

10. The block copolymer of claim 1, wherein the block copolymer has a Zeta potential (mV) of about 30 to about 65.

11. A polymer nanoparticle comprising a block copolymer of claim 1.

12. The polymer nanoparticle of claim 11, wherein the nanoparticle has a PDI of about 0.05 to about 0.6, as measured by DLS.

13. A composition comprising:
a polymer nanoparticle according to claim 11, and
a nucleic acid complexed to the polymer nanoparticle.

14. The composition of claim 13, wherein the nucleic acid is RNA.

15. The composition of claim 13, wherein the nucleic acid complexed to the polymer nanoparticle transfects a cell.

16. The composition of claim 13, wherein the composition has a cell transfection efficiency of about 50% to about 99%.

17. The composition of claim 13, wherein the cell is a mammalian cell.

18. The composition of claim 13, wherein the cell is a neuronal cell.

19. A method of treating a disease in a patient in need thereof, the method comprising administering a composition according to claim 13.

20. The method of claim 19, wherein the polymer nanoparticles are delivered to the brain of the patient.
